(19) United States
(12) Patent Application Publication
(10) Pub. No.: US 2014/0000648 A1
(43) Pub. Date: Jan. 2, 2014

(54) System and method for cleaning and sanitizing mattresses

(71) Applicant: Michael Ingle, Corinth, TX (US)

(72) Inventor: Michael Ingle, Corinth, TX (US)

(21) Appl. No.: 13/507,425

(22) Filed: Jun. 27, 2012

(51) Int. Cl. B08B 3/00

(52) U.S. Cl. 134/1; 134/105

(57) ABSTRACT

The invention disclosed herein is a system and method for cleaning and sanitizing mattresses infested with insects and bacteria. Specifically, mattresses infested with bed bugs and/or dust mites may be cleaned and sanitized through a series of steps beginning with UV irradiation of the mattress. Next, the mattresses are subject to dry steam of a temperature of approximately 356° F. The mattress is then vacuumed to remove debris and waste. Next the mattress is heated with infrared heaters which produce a temperature of up to 265° F. Finally, the mattress is again vacuumed to remove any remaining debris. The system is mounted on an enclosed trailer so that the system may be transported to a home, hospital, hotel or dormitory to clean and sanitize the mattresses.

### CROSS-REFERENCE TO RELATED APPLICATIONS

None

### STATEMENT REGARDING FEDERALLY SPONSORED RESEARCH OR DEVELOPMENT

No part of the invention disclosed herein was the subject of federally sponsored research or development.

### THE NAMES OF THE PARTIES TO A JOINT RESEARCH AGREEMENT

None

### REFERENCE TO A SEQUENCE LISTING

Not applicable.

### BACKGROUND OF THE INVENTION

1. Field of the Invention

The field of the invention disclosed herein is the cleaning and sanitizing of mattresses in both the commercial and residential setting. The invention disclosed herein is a mobile system incorporating ultraviolet light, dry steam, vacuum suction and infrared heat to kill and remove insects, bacteria and debris commonly found on residential and commercial bed mattresses.

2. Description of the Prior Art

Mattresses have been developed to enhance the comfort enjoyed by the individual while sleeping in bed. Mattresses are made from many different materials and may be of different sizes as well. With use, mattresses can become dirty and infested with parasites such as dust mites and bed bugs as well as bacteria such as Staphylococcus. Dust mites are microscopic insects related to other mites, and ticks, in the class Arachnida, which also includes spiders, scorpions, harvestmen (daddy-longlegs) and similar eight-legged creatures. Classification of mites is ever evolving but are currently placed in the order Astigmata, family Pyroglyphidae, and genus Dermatophagoides. The two most common species are 1) Dermatophagoides farina, the North American house dust mite and 2) Dermatophagoides pteronyssinus, the European house dust mite. Both species inhabit dwellings where humans live and where the humidity is optimal for the mite life cycle. Dust mites are infamous for causing allergies in people who sleep on mite infested mattresses. A protein in the mite fecal pellet causes asthma, or aggravates existing asthma symptoms. Dust mite material can also cause eczema whereby the skin becomes dry, itchy, red and scaly. One of the obstacles to removing mites from infested mattresses is that they are literally too small to be seen with the naked eye. Adult dust mites range in size from 250-300 microns and dust mite larvae and nymphs are even smaller making them invisible to the naked eye. Dust mites can be seen with a microscope, but such an instrument is not commonly found in the home or in a commercial setting such as a hotel, dormitory, nursing home and/or hospital. Further, microscopes require specific training on the part of the operator in order to be used properly. Dust mites can infest a used mattress in enormous numbers. Typically, the number of dust mites in an infested mattress range from 100,000 to ten million per mattress. Dust mites feed almost exclusively on dead skin cells which people shed daily by the thousands. Mattresses are the chief place where the majority of house dust mites reside. However, mites can also be found on pillows, overstuffed furniture, even rugs and carpeting. Mites can also be found where household pets sleep.

In addition to dust mites inhabiting mattresses, bed bugs can also infest mattresses. Bed bugs feed on the blood of people. The bed bug will bite the victim and ingest the victim's blood. Bed bugs can live for more than a year without feeding. The bites themselves are painless, but may leave itchy, red scars which are apparent the next morning. Typical symptoms of bed bug bites are small red spots on the skin in rows of three or more. Some people who are bitten by bed bugs demonstrate no symptoms whatsoever, while others can have severe skin reactions. People become increasingly sensitive to bed bug bites with repeated exposure to bed bug bites over time. Bed bugs are much larger in size than dust mites ranging in size from 1.5-7 millimeters. At this size, the bed bugs are visible to the naked eye and visual inspection of an infested mattress may reveal the presence of bed bugs but not dust mites because of their small size. Bed bugs live on mattresses but are only active at night. During the day the bed bug may hide, often under the buttons on mattresses, or in the seams along the edge of the mattress. Bed bugs can also be found in the slots formed by the bed frame. Bed bugs have also been found behind headboards, pictures and posters on the wall and behind peeling wallpaper, among other locations. In contrast dust mites are small enough to slip easily between the threads in the fabric of a mattress pad or the mattress itself.

In addition to bed bugs, dust mites and other insects, the mattress may be contaminated with bacteria such as those of the genus Staphylococcus. These bacteria are classified as Gram positive bacteria and are resistant to drying. Staphylococci are small in size (1-3 microns) and are present on the skin of most people in enormous numbers. The bacteria are shed from the skin of an individual onto the mattress where the bacteria can survive for some time. Bacteria such as Staphylococcus can cause a broad spectrum of illness ranging from abscesses and pustules to blood infection and death. These organisms are also becoming alarmingly resistant to many antibiotics.

Efforts have been undertaken to remove the insects and bacteria from infested mattresses. Efforts to remove bacteria have met with limited success mainly because of the enormous numbers of bacteria present on the mattress. To remove bacteria from a mattress, the mattress is usually scrubbed with an antibacterial agent which is allowed to remain in contact with the mattress until the antibacterial agent has evaporated and the mattress is dry. This method of decontamination does not eliminate the bacteria but reduces the number of bacteria by about a factor of 10.

Efforts to remove insects such as bed bugs and dust mites have centered around the use of special protective covers which are placed on the mattress to separate the insects from the sleeping individual. Clearly, this method acts only as a barrier to contact between the insects and the individual and does not kill or remove the insects from the mattress. Removal of dust mites is critical to preventing illness from dust mites because it is the digestive proteins found in the fecal pellet which causes illness and may be allergenic long after the dust mite has expired. What is needed in the art is a system and method which kills and removes insects and bacteria from a mattress.

### BRIEF DESCRIPTION OF THE INVENTION

The invention disclosed herein is a system and method which kills and removes insects and bacteria from a mattress. Specifically, the system and method which kills and removes insects and bacteria from a mattress is composed of several steps. The first step is to irradiate the mattress in UV light. The second step in the system and method disclosed herein is to clean the mattress with dry steam using distilled water of approximately 356° F. The third step is to remove any unwanted waste and debris left behind by the bed bugs and mites by a vacuum plenum. The fourth step is to heat the mattress with infrared heaters up to a temperature of 265° F. to dry any moisture created by the dry steam while killing any bed bugs and dust mites which may have survived the previous three steps. The fifth step is again a vacuum step to remove any remaining debris. The aforementioned UV lights, dry steam apparatus, vacuums and infrared heaters are mounted to an enclosed frame which is mounted to a box van or truck cab chassis or enclosed trailer which can be transported to a home, hospital, dormitory, hotel or other location where mattresses need to be cleaned and sanitized. Electric power is supplied to the components of the system and method by a gas or diesel powered generator. The mattress to be cleaned and sanitized is placed inside the trailer and the devices are activated and do their assigned functions automatically.

### BRIEF DESCRIPTION OF THE DRAWING/FIGURES

A better understanding of the system and method for cleaning and sanitizing mattresses may be had by examination of the following drawings/figures:

FIG. 1 is a top view of the system and method for cleaning and sanitizing mattresses;

FIG. 2 is an end view of the system and method for cleaning and sanitizing mattresses;

FIG. 3 is a side view of the system and method for cleaning and sanitizing mattresses;

FIG. 4 is a an end view opposite that of FIG. 2 of the system and method for cleaning and sanitizing mattresses;

### DETAILED DESCRIPTION OF THE INVENTION

The invention disclosed herein is a system and method for cleaning and sanitizing mattresses which in one embodiment consists of ultraviolet lights, dry steam, vacuum, infrared heaters and an electrical power source such as a gas or diesel powered generator. In a preferred embodiment, the ultraviolet lights, dry steam, vacuum, infrared heaters are situated in a contained movable enclosure such as an enclosed trailer capable of being towed by a car or pickup truck. The components of the system to clean and disinfect mattresses may also be contained within a closed truck or van for transport to the work site. The electrical generator is situated outside of the truck or trailer enclosure. An opening in the side of the truck or trailer enclosure permits the operator to insert the mattress inside the enclosure containing the components of the cleaning and sanitizing system. The operator inserts the mattress and starts the generator which provides power to the cleaning and sanitizing system. A conveyor system moves the mattress to each of the components of the cleaning and sanitizing system.

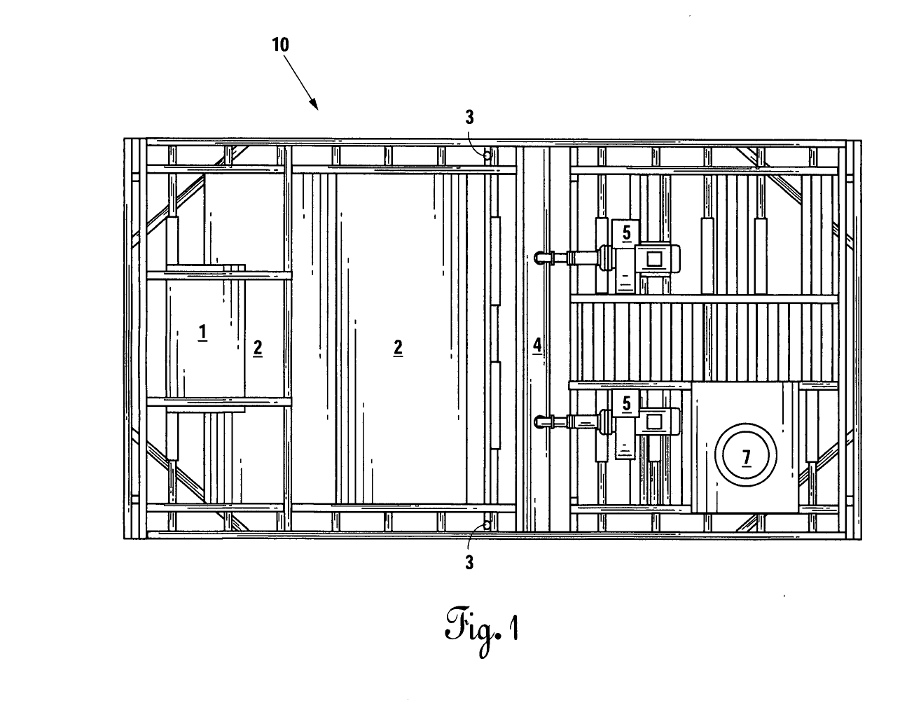

As shown in FIG. 1 the cleaning and sanitizing system 10 disclosed herein is situated in an box van or cab chassis. The UV light assemblies 2 receive power from the UV light assembly power supply 1. The UV light assemblies 2 are mounted to be approximately 2-6 inches from the mattress. In the embodiment of the system and method disclosed herein disclosed in FIG. 1, 26 UV light assemblies 2 are mounted approximately 2 to 6 inches away from the mattress and configured so as to irradiate the entire mattress for approximately six minutes with UV light of approximately 260 nanometers wavelength. Bed bugs are susceptible to UV light in the 253 nanometer wavelength The dry steam spray manifold 3 receives steam of approximately 356° F. from a steam generator 7. Bed bugs are destroyed by a temperature of 113° F. The mattress to be sanitized and cleaned is dry steamed by the steam from the dry steam spray manifold 3 for approximately 6 minutes. The dry steam manifold 3 is located approximately 3 inches from the mattress. A vacuum plenum 4 almost in contact with the mattress removes waste and debris from the mattress which is then ejected from the enclosed trailer through suction fans 5. In the embodiment of the system and method shown in FIG. 1, two separate vacuum plenums 3 each with 1.5 horsepower are used. These vacuum plenums 3 have a vacuum of 1280 cubic feet per minute each. The mattress moves from the vacuum plenums 3 to a bank of infrared heaters 6 which are located approximately 18 inches away from the mattress. The mattress stays under the infrared heaters for approximately 6 minutes. The mattress to be cleaned and sanitized is inserted into the system 10 and a conveyor belt (not shown) moves the mattress to the various components of the system and method 10 including a series of infrared heaters (not shown). The mattress is raised or lowered to the proper height by a series of mattress height compensator actuators (not shown). The entire cycle for cleaning and disinfecting a mattress is approximately 12 minutes.

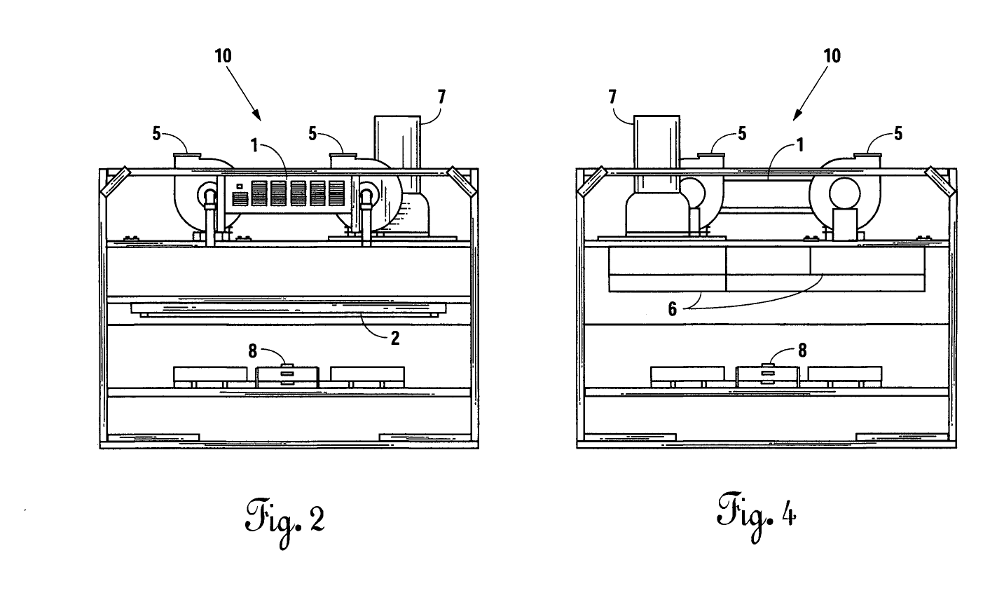

The conveyor system 8 is shown in FIGS. 2 and 4. The mattress to be cleaned and sanitized is placed on the conveyor system 8 which initially positions the mattress underneath the UV light assemblies 2. The UV light assemblies 2 receive power from the UV light power supply 1. The suction fans 2 which exhaust debris captured by the vacuum plenums (not shown) are oriented so as to direct the debris to the outside of the enclosure. As shown in FIG. 4, the infrared heaters 6 generate up to 5120 BTU's of heat which produces temperatures of up to 265 degrees. The infrared heaters 6 will evaporate any moisture created by the dry steam while also killing any surviving bed bugs and dust mites.

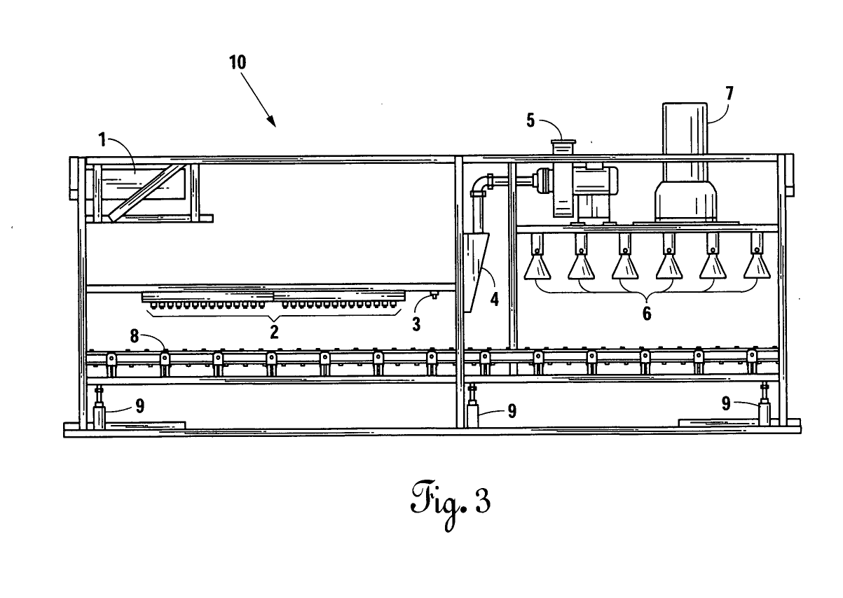

The orientation of the components of the system and method for cleaning and sanitizing mattresses 10 is shown in FIG. 3. The mattress to be cleaned and sanitized is placed on the conveyor system 8 underneath the UV light assemblies 2 which draw their power from the UV light power supply 1. The conveyor system 8 is raised or lowered by mattress height compensator actuators 9 so that the top of the mattress is approximately 2-6 inches from the UV light assemblies 2. The mattress to be cleaned and disinfected is continuously moved by the conveyer belt 8 so that when the UV light cycle is initiated, the mattress will start moving to the dry steam manifolds 3 which will cleanse the mattress by dry steam obtained from the steam generator 7. The mattress height compensator actuators adjust the height of the conveyor system 8 so that the mattress is approximately 3 inches from the dry steam manifolds 3. As the mattress leaves the dry steam manifolds 3, the conveyor system 8 is raised by the mattress height compensator actuators 9 so that the mattress is almost in contact with the vacuum plenums 4. The mattress is vacuumed by the vacuum plenums 4 for removal of waste and debris from the surface of the mattress. Upon leaving the vacuum plenums 4, the mattress is moved by the conveyor system 8 to a position underneath the infrared heaters 6. The infrared heaters 6 generate a heat of up to 265° F. to evaporate any moisture remaining from the dry steam cycle and killing any remaining insects. The mattress is underneath the infrared heaters for approximately six minutes. The mattress height compensator actuators 9 position the conveyor system 8 so that the mattress is approximately 18 inches from the infrared heaters 6. Finally, the conveyor system 8 moves the mattress from underneath the IR heaters 6 to the vacuum plenums 4 for removal of any remaining debris. The mattress height compensator actuators 9 raise the conveyor system 8 so that the mattress is almost in contact with the vacuum plenums 3. After passing under the vacuum plenums 4, the mattress is moved to the starting position underneath the UV lights 2. The power is turned off and the mattress is removed from the enclosure. The entire cleaning and disinfecting cycle takes about 12 minutes. At the conclusion of the cleaning and sanitizing procedure, the mattress is cleaner and free of observable bed bugs or dust mites.

The system and method for cleaning and sanitizing mattresses disclosed herein is susceptible to many different embodiments. The size and number of the various components can vary to accomplish the same goals with respect to other items such as pillows, blankets, chair and sofa cushions, dog beds, clothing, luggage and a myriad of other items. All of these other embodiments are incorporated into this specification as limited by the claims appended hereto.

I claim:

1. A system for sanitizing and cleaning mattresses infested with insects and bacteria comprising:
a source of electrical power;
a plurality of UV light assemblies which emit UV light in approximately the wavelength of 260 nanometers;
a source of dry steam which steams said mattress with steam of approximately 356° F.;
a plurality of vacuum sources;
a source of infrared heat which generates heat of approximately 265° F.;
a conveyor system to move said mattress from said UV light assemblies to said source of dry steam to said source of vacuum to said source of infrared heat to said source of vacuum;
a plurality of mattress height compensator actuators to position said mattress at the correct height under said UV light assemblies, said source of dry steam, said source of vacuum and said source of infrared heat;
Whereby said insects are killed and removed from the mattress.

2. The system for sanitizing and cleaning mattresses infested with insects and bacteria as defined in claim 1 wherein said insect is a bed bug.

3. The system for sanitizing and cleaning mattresses infested with insects and bacteria as defined in claim 1 wherein said insect is a dust mite.

4. A method for sanitizing and cleaning mattresses infested with insects and bacteria comprising steps for:
Irradiating said mattress with UV light located approximately 2-6 inches from said mattress of a frequency of approximately 260 nanometers for approximately 6 minutes from a plurality of UV light sources;
steaming said mattress with steam located approximately 3 inches from said mattress at a temperature of approximately 356° F. for approximately 6 minutes;
vacuuming said mattress with vacuums which are nearly touching said mattress and which produce a vacuum of approximately 1280 cubic feet per minute each;
heating for approximately 6 minutes said mattress with infrared heat of approximately 265° F. located approximately 18 inches away from said mattress for approximately 6 minutes;
vacuuming said mattress with vacuums which are nearly touching said mattress and which produce a vacuum of approximately 1280 cubic feet per minute each.

5. The method for cleaning and sanitizing mattresses infested with insects and bacteria as defined in claim 4 wherein said insect is a bed bug.

6. The method for cleaning and sanitizing mattresses infested with insects and bacteria as defined in claim 4 wherein said insect is a dust mite.
